(19) United States
(12) Patent Application Publication
(10) Pub. No.: US 2025/0375546 A1
(43) Pub. Date: Dec. 11, 2025

(54) A Trapping Device And Implementations Thereof

(71) Applicants: Indian Institute of Science, Bangalore (IN); The Regents of the University of California, Oakland, CA (US)

(72) Inventors: Deepak D. Patil, Bangalore (IN); Kaushik Chatterjee, Bangalore (IN); Maya R. Overland, San Francisco, CA (US); Marshall Stoller, San Francisco, CA (US)

(21) Appl. No.: 18/877,161

(22) PCT Filed: Jun. 29, 2023
(86) PCT No.: PCT/IN2023/050632

§ 371 Date: Dec. 19, 2024

(30) Foreign Application Priority Data
Jun. 29, 2022 (IN) 202241037423

(51) Int. Cl. A61L 2/00; A61F 2/01; A61K 9/00; A61K 38/12; A61L 101/02

(52) U.S. Cl. CPC A61L 2/0082; A61F 2/011; A61K 9/0034; A61K 38/12; A61F 2240/001; A61L 2101/02

(57) ABSTRACT

The present disclosure provides a trapping device for trapping microbes, the device comprises a porous polymeric structure with a proximal end and a distal end, wherein the proximal end and the distal end is optionally provided with a hole or core on the 5 longitudinal direction. The present disclosure also provides a process of preparing the device and implementations thereof.

### FIELD OF INVENTION

The subject matter of the present disclosure broadly relates to porous polymeric structure and particularly, relates to a trapping device comprising the porous polymeric structure. Additionally, the present disclosure relates to trapping of microbes by the trapping device.

### BACKGROUND OF THE INVENTION

Growing antibiotic resistance patterns related to overuse of antibiotics in humans and in livestock are an urgent threat to public health. World Health Organization identified antimicrobial resistance as a high-priority economic development and global health challenge. The rise in new antibiotic resistance is faster than the development of novel pharmaceutical agents. Progressively increasing antibiotic resistances with recurrent infections require escalation to broader spectrum antibiotics with side effect profiles including disruption of the systemic microbiome and increased risk of opportunistic infections such as Clostridium difficile.

For instance, urinary tract infections (UTIs) place a huge burden upon patients and the healthcare system with an estimated annual cost in the United States alone of over $2.3 billion, with an additional $2.9 billion in direct and indirect costs for admissions for acute pyelonephritis. In the nursing home, the annual incidence of UTI in women over 85 years of age is up to 29.6%. An indwelling urinary catheter increases the risk of bacteriuria by 3-10%. It has been demonstrated that urine is not sterile but rather that a urinary microbiome exists and may play a role in other disease processes, including urolithiasis, incontinence, and interstitial cystitis. Suppressive antibiotics given at reduced dosages and at lower frequencies than therapeutic antibiotics are very effective at decreasing the incidence of UTI by decreasing bacterial colonization, however, this comes with a high risk of building resistance.

Therefore, there is an emerging need for an alternate and effective approach towards inhibiting, or reducing the bacterial load in an environment which will prove to be beneficial in all fields, especially in the field of biomedical applications.

### SUMMARY OF THE INVENTION

In an aspect of the present disclosure, there is provided a trapping device for trapping microbes, the device comprises a porous polymeric structure with a proximal end and a distal end, wherein the proximal end and the distal end is optionally provided with a hole or core on the longitudinal direction.

In second aspect of the present disclosure, there is provided a process for preparing the device comprising a porous polymeric structure with a proximal end and a distal end, wherein the proximal end and the distal end is optionally provided with a hole or core on the longitudinal direction, the process comprising: a) mixing a first polymer solution comprising a biocompatible polymer selected from polycaprolactone, polylactic acid, or combinations thereof with a porogen optionally in the presence of silver nanoparticles and an antimicrobial agent or an additive to obtain a first solution; b) vacuum drying the first solution to obtain a polymeric structure; and c) molding the polymeric structure to obtain the device.

In third aspect of the present disclosure, there is provided a method of treating or preventing a disease or a condition or an infection caused by a microbe, the method comprising: a) inserting the device comprising a porous polymeric structure with a proximal end and a distal end, wherein the proximal end and the distal end is optionally provided with a hole or core on the longitudinal direction with a delivery tool into urinary bladder through urethra; b) releasing the device into the urinary bladder and removing the delivery tool; c) allowing the device to trap the microbes; and d) reinserting the delivery tool into the urinary bladder and retracting the device.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

FIG. 1 depicts A) photographs of neat polycaprolactone (A1) solution; (A2) neat PCL structure with pores size range 125-250 μm, and (B) representative scanning electron microscopic (SEM) images of microporous neat polycaprolactone structure of (B1 and B2) pore size 125-250 μm, and (C1 and C2) pore size 125-350 μm, in accordance with an implementation of the present disclosure.

FIG. 2 depicts A) photographs polycaprolactone solution with increasing silver nanoparticles concentration from left to right, and (B) photographs of the microporous polycaprolactone structure with varying silver nanoparticles, in accordance with an implementation of the present disclosure.

FIG. 3 depicts A) series of the polymeric structure comprising polycaprolactone; and B) series of the polymeric structure comprising polycaprolactone with silver nanoparticles (AgNPs), in accordance with an implementation of the present disclosure.

FIG. 4 depicts (A) scanning electron micrographs of porous polycaprolactone (A1) top surface (A2) bottom surface, (B) the cross-sectional view of porous polycaprolactone embedded with silver nanoparticles (AgNPs) shown in magnified images, (C) energy-dispersive X-ray (EDS) spectra confirming the presence of AgNPs, (D) porous polycaprolactone structure without AgNPs trapping E. coli (Escherichia coli) in interconnected pores as seen in the magnified image, (E) several dead E. coli observed on porous polycaprolactone structure containing AgNPs, in accordance with an implementation of the present disclosure.

FIG. 5 depicts the optical density value for the E. coli suspension in contact with the porous polycaprolactone structure and porous polylactic acid structure of different pore sizes, in accordance with an implementation of the present disclosure.

FIG. 6 depicts optical density (OD) values of the E. coli suspension in contact with neat macro-porous PCL of various pore sizes in comparison to blank and non-porous PCL samples (initial OD value: 0.04), (all data are shown as mean±S.D. for n=3), in accordance with an implementation of the present disclosure.

FIG. 7 depicts SEM micrographs of (A) NaCl crystals (125-250 μm), (B) the corresponding porous PCL structure, (C) crushed NaCl crystals (45-63 μm) and (D) the corresponding porous PCL structure fabricated from crushed NaCl crystals, in accordance with an implementation of the present disclosure.

FIG. 8 depicts (A) photograph of customized reactive ion etching (RIE) system, (B) SEM images of macroporous PCL sample etched using RIE, and (C) dose dependent effect of RIE on E. coli clearance from culture solution over time, in accordance with an implementation of the present disclosure.

FIG. 9 depicts a plot of dose dependent effect of RIE on E. coli clearance after 9 hours, in accordance with an implementation of the present disclosure.

FIG. 10 depicts the optical density value for the E. coli suspension in contact with the porous polycaprolactone structure loaded with varying concentrations (in mg/ml) of silver nanoparticles, in accordance with an implementation of the present disclosure.

FIG. 11 depicts the optical density value for the E. coli suspension in contact with the porous polycaprolactone structure loaded with silver nanoparticles (AgNPs) for a time period of 24 hours and 48 hours, in accordance with an implementation of the present disclosure.

FIG. 12 depicts bacterial colony count of cells for E. coli suspension in contact with porous PCL loaded with different AgNPs concentration after various incubation times, in accordance with an implementation of the present disclosure.

FIG. 13 depicts (A) porous PCL, (B) CuNPs (copper nanoparticles) incorporated porous PCL, (C, D and E) electron micrographs of CuNPs incorporated porous PCL, in accordance with an implementation of the present disclosure.

FIG. 14 depicts release of silver ion and copper ions measured using atomic absorption spectroscopy in phosphate buffer solution (PBS) till 24 h, in accordance with an implementation of the present disclosure.

FIG. 15 depicts optical density values for (A) E. coli, (B) S. aureus (Staphylococcus aureus), and (C) P. aeruginosa (Pseudomonas aeruginosa) bacterial culture suspensions in contact with porous PCL (125-250 μm pore sizes) either neat or etched on one side or loaded with AgNP concentrations of 5 mg/mL, in accordance with an implementation of the present disclosure.

FIG. 16 depicts the device comprising the molded porous polymeric structure, in accordance with an implementation of the present disclosure.

FIG. 17 depicts the device comprising the porous polymeric structure with a suture, in accordance with an implementation of the present disclosure.

FIG. 18 depicts the device comprising the porous polymeric structure with a string, in accordance with an implementation of the present disclosure.

FIG. 19 depicts the device comprising the porous polymeric structure with a suture and a string, in accordance with an implementation of the present disclosure.

FIG. 20 depicts representative images of the device in a A) folded; B) semi-folded; and C) wave shaped structures, in accordance with an implementation of the present disclosure.

FIG. 21 depicts (a) optical micrograph of Teflon mold and porous PCL with suture embedded, representative SEM images of porous PCL rope (b) top surface and (c) cross-sectional surface, in accordance with an implementation of the present disclosure.

### DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “trapping device” used herein refers to a device which is capable of capturing the microbes from any media including a fluid, wherein the device is in contact with the fluid which is containing microbes. The trapping device can be made in contact with the fluid either by immersing the device in the fluid or by allowing the fluid to pass through the device. The device captures the microbes, kills the microbes and further inhibits the growth of the microbes. The trapping device of the present disclosure comprises porous polymeric structure with a proximal end and a distal end, wherein the proximal end and the distal end is optionally provided with a hole or core on the longitudinal direction. The device comprises one or more of the polymeric structures to form plurality of the porous structure and the plurality of the porous polymeric structures are arranged either linearly or in a complex folded structure for deployment in any required shapes. The trapping device of the present disclosure is a mechano-bactericidal device capable of trapping and/or killing the microbes. The porous polymeric structure is incorporated with silver nanoparticles which provides bactericidal property to the device.

The term “porosity” used herein refers to a measure of pores present in a unit area. The porous polymeric structure of the present disclosure has porosity in a range of 70 to 98%.

The term “antimicrobial agent” used herein refers to a substance, a chemical, or a material which can kill or inhibit the growth of the microorganism. The antimicrobial agent includes but not limited to metal particles, metal oxide particles, antibiotics, cationic polymers, or combination thereof. The metal particles includes but not limited to Au, or Cu particles; and the metal oxide particles includes but not limited to CuO, ZnO, TiO₂, and Fe₃O₄.

The term “additive” used herein refers to a substance optionally added in the polymeric structure to provide specific characteristics to the device. For example, the additive is selected from a toxin, an attractants, or a chelating agent. The chelating agent includes but not limited to polymyxin B which is capable of binding endotoxin.

The term “biocompatible polymer” used herein refers to the polymeric material which are non-toxic to biological tissues. In the present disclosure, the biocompatible polymer includes, but is not limited to polyurethane, polyethylene, polypropylene, polycaprolactone, polylactic acid, polystyrene, silicone, polysaccharide, or combinations thereof. The term “silicone” refers to the inorganic silicone polymers which are of medical grade. The term “polysaccharide” include but not limited to chitosan and so on.

The term “delivery tool” or “a retrieval tool” used herein refers to a tool or a device or an instrument used to insert or retrieve or retract the device of the present disclosure. The delivery tool of the present disclosure is an accessory that can be attached to the device to deliver the device at the requisite areas. The delivery tool of the present disclosure shall be used to insert the device into the urethra through urinary bladder and/or retract the device from the urinary bladder after trapping of the microbes by the device. The delivery tool of the present disclosure includes but not limited to a catheter, a device deposited via direct visualization into the lumen with an endoscopic cytoscope and the retrieval tool is a hook type device, a magnetic retrieval device, or an endoscope with a grasper.

The term “porogen” used herein refers to materials that are used to create porous structures, which are inert and does not react to the substance to which the porogens are added. These porogens creates voids and pores of specific shape and size. The porogen of the present disclosure includes but not limited to sodium chloride, paraffin spheres, sugar, or gelatin.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a porosity range of 70 to 98% should be interpreted to include not only the explicitly recited limits of 70 to 98%, but also to include subranges, such as 71 to 97%, 75 to 90% and so forth, as well as individual amounts, including fractional amounts, within the specified ranges, such as 70.5%, 82.3% and 95.8%, for example.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As discussed in the background, rise of antimicrobial resistance (AMR) has led to an increased focus on developing strategies for tackling bacterial infections. Physical and mechanical approaches provides a route that is independent of antibiotic-based strategies. The present disclosure uses mechanical approach to trap the microbes and to eliminate the microbes through trapping, infiltration and by microbial cell death. The present disclosure utilizes mechanical entrapment of bacteria to reduce bacterial load in a liquid suspension using a porous foam. The antibacterial activity of the engineered trap is further augmented by introducing mechanobactericidal topography and/or incorporation of bactericidal chemicals/biochemicals in the trap. The present disclosure provides a mechanical trap for bacterial cells which are easily deployable and removable to sequester and reduce the load of suspended bacteria. Accordingly, the present disclosure provides a trapping device comprising a porous polymeric structure with a proximal end and a distal end, wherein the proximal end and the distal end is optionally provided with a hole or core on the longitudinal direction. The device of the present disclosure has one or more of the polymeric structures which are in simple linear arrangement or is in complex folded arrangement. The device of present disclosure is adaptable to integrate nanoscale topographic features on the surface of the polymeric foam and to incorporate antimicrobial agents, toxins, attractants, or chelating agent. Further the device of the present disclosure are adaptable to any geometry and size for trapping bacterial cells.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a trapping device for trapping microbes, the device comprises a porous polymeric structure with a proximal end and a distal end.

In an embodiment of the present disclosure, there is provided a trapping device for trapping microbes, the device comprises a porous polymeric structure with a proximal end and a distal end, wherein the proximal end and the distal end is optionally provided with a hole or core on the longitudinal direction.

In an embodiment of the present disclosure, there is provided a trapping device for trapping microbes as disclosed herein, wherein the polymeric structure has a breadth to length ratio in a range of 1:50 to 10:200, a porosity in a range of 70 to 98% and pore size in a range of 10 to 500 μm.

In an embodiment of the present disclosure, there is provided a trapping device for trapping microbes as disclosed herein, wherein the device comprises one or more of the polymeric structure and is arranged linearly or in a complex folded shape for deployment.

In an embodiment of the present disclosure, there is provided a trapping device for trapping microbes, the device comprises a porous polymeric structure in the form of a sheet.

In an embodiment of the present disclosure, there is provided a trapping device for trapping microbes as disclosed herein, wherein the polymeric structure has a breadth to length ratio in the range of 1:50 to 10:200, a porosity in the range of 70 to 98% and pore size in the range of 10 to 500 μm and is arranged either as a simple linear cord or in a more complex folded shape for deployment.

In an embodiment of the present disclosure, there is provided a trapping device for trapping microbes, the device comprises one or more porous polymeric structure with a proximal end and a distal end, the polymeric structure having a breadth to length ratio in the range of 1:50 to 10:200, a porosity in the range of 70 to 98% and pore size in the range of 10 to 500 μm, wherein the proximal end and the distal end is optionally provided with a hole or core on the longitudinal direction; and the polymeric structure is arranged linearly or is folded to a shape for deployment.

In an embodiment of the present disclosure, there is provided a trapping device for trapping microbes as disclosed herein, wherein the polymeric structure is incorporated with silver nanoparticles.

In an embodiment of the present disclosure, there is provided a trapping device for trapping microbes as disclosed herein, wherein the device comprises one or more antimicrobial agent, or an additive embedded within the polymeric structure or grafted on to the surface of the walls of the trapping device.

In an embodiment of the present disclosure, there is provided a trapping device for trapping microbes as disclosed herein, wherein the antimicrobial agent is selected from metal particles, metal oxide particles, antibiotics, cationic polymers, or combinations thereof; and the additive is selected from chelating agent, toxins, attractants, or combinations thereof.

In an embodiment of the present disclosure, there is provided a trapping device for trapping microbes as disclosed herein, wherein the additive is chelating agent, toxins, attractants, or combinations thereof, the chelating agents includes but not limited to penicillamine for Cu²⁺, N,N,N′,N′-tetrakis(2-pyridylmethyl)-ethylenediamine for Zn²⁺, or deferoxamine and gramibactin for Fe³⁺; the attractants includes but not limited to sugars such as maltose, ribose, and galactose, and amino acids like L-aspartate and L-serine; and the toxins includes but not limited to ligands, cholera toxin, eicosanoid receptor, G protein, enzymes, antigen, peptide, and proteome.

In an embodiment of the present disclosure, there is provided a trapping device for trapping microbes as disclosed herein, wherein the metal particles is selected form Au, Cu, or combinations thereof; and the metal oxide particles is selected from CuO, ZnO, TiO₂, Fe₃O₄, or combinations thereof.

In an embodiment of the present disclosure, there is provided a trapping device for trapping microbes as disclosed herein, wherein the chelating agent is capable of binding endotoxin, and the chelating agent includes but not limited to polymyxin B.

In an embodiment of the present disclosure, there is provided a trapping device for trapping microbes as disclosed herein, wherein the device comprises a suture inserted between the proximal end to the distal end.

In an embodiment of the present disclosure, there is provided a trapping device for trapping microbes as disclosed herein, the device comprises a porous polymeric structure with a proximal end and a distal end; and a suture inserted between the proximal end to the distal end, wherein the proximal end and the distal end is optionally provided with a hole or core on the longitudinal direction.

In an embodiment of the present disclosure, there is provided a trapping device for trapping microbes as disclosed herein, wherein the polymeric structure is made of a biocompatible polymer selected from polycaprolactone, polylactic acid, chitosan, or combinations thereof.

In an embodiment of the present disclosure, there is provided a trapping device for trapping microbes as disclosed herein, wherein the suture is obtained from a biocompatible material selected from polyurethane, polyethylene, polypropylene, polycaprolactone, polylactic acid, polystyrene, silicone, nylon, polydioxine, polysaccharide, or combinations thereof. In another embodiment of the present disclosure, the suture is a biocompatible material selected from polyurethane, polyethylene, polypropylene, polycaprolactone, polylactic acid, polystyrene, silicone, polysaccharide, or combinations thereof.

In an embodiment of the present disclosure, there is provided a trapping device for trapping microbes as disclosed herein, wherein the suture has breadth and length in a ratio range of 0.2:50 to 0.5:200; and the suture has the length in a range of 5 to 20 cm, and the breadth in a range of 0.2 to 0.5 mm.

In an embodiment of the present disclosure, there is provided a trapping device for trapping microbes as disclosed herein, wherein the device further comprises a string attached to the device or the suture.

In an embodiment of the present disclosure, there is provided a trapping device for trapping microbes as disclosed herein, wherein the device further comprises a string attached to the device at the proximal end.

In an embodiment of the present disclosure, there is provided a trapping device for trapping microbes as disclosed herein, wherein the device further comprises a string attached to the device; and the string is the suture that is left long, or the string is an extension of the suture.

In an embodiment of the present disclosure, there is provided a trapping device for trapping microbes as disclosed herein, wherein the device is an implantable or an insertable device.

In an embodiment of the present disclosure, there is provided a trapping device for trapping microbes as disclosed herein, the device comprises a porous polymeric structure with a proximal end and a distal end, a suture inserted between the proximal end to the distal end and a string, wherein the proximal end and the distal end is optionally provided with a hole or core on the longitudinal direction; and the string is the suture that is left long or the string is an extension of the suture.

In an embodiment of the present disclosure, there is provided a trapping device for trapping microbes as disclosed herein, wherein the device comprises a delivery tool at the proximal end adapted to retrieve the device.

In an embodiment of the present disclosure, there is provided a trapping device for trapping microbes as disclosed herein, wherein the device acts as a mechanical trap for a microbe.

In an embodiment of the present disclosure, there is provided a trapping device for trapping microbes as disclosed herein, wherein the device acts as a mechanical trap for a microbe selected from bacteria, fungi, or virus.

In an embodiment of the present disclosure, there is provided a trapping device for trapping microbes as disclosed herein, wherein the device acts as a mechanical trap for a microbe selected from bacteria, fungi, or virus; and the microbe is selected from Escherichia coli, Pseudomonas aeruginosa, Helicobacter pylori, Salmonella species, Vibrio cholera, Klebsiella, Staphylococcus, Enterococcus or uropathogens.

In an embodiment of the present disclosure, there is provided a trapping device for trapping microbes as disclosed herein, wherein the device is one or more polymeric structure which can be molded to various shapes and sizes; and the device shall be adaptable for easy deployment in any area of microbial load.

In an embodiment of the present disclosure, there is provided a trapping device for trapping microbes as disclosed herein, wherein the device has a surface texture in microscale, nanoscale, or combination of micro-nano scale; and the surface texture is achieved by means of top-down or bottom-up approach; and the device is fabricated into various surface textures to avoid biofilm formation on the surfaces. In another embodiment of the present disclosure, the device is subjected to reactive ion etching and the device has a surface texture in nanoscale.

In an embodiment of the present disclosure, there is provided a trapping device for trapping microbes as disclosed herein, wherein the surface texture ranges from 10 nm to 1000 nm in height and in spacing.

In an embodiment of the present disclosure, there is provided a trapping device for trapping microbes as disclosed herein, wherein the device has the polymeric structure with the surface texture optimized to allow for the application of insonation to maximize bacterial cell death at the surface-urine interface.

In an embodiment of the present disclosure, there is provided a process for preparing the trapping device for trapping microbes as disclosed herein, the process comprising: a) mixing one or more of a biocompatible polymer selected from polycaprolactone, polylactic acid, or combinations thereof with a porogen, optionally in the presence of silver nanoparticles, an antimicrobial agent, or an additive to obtain a first solution; b) vacuum drying the first solution to obtain a polymeric structure; and c) molding the polymeric structure to obtain the device.

In an embodiment of the present disclosure, there is provided a process for preparing the trapping device for trapping microbes as disclosed herein, wherein the biocompatible polymer is chitosan, which upon freeze drying, result in the polymeric structure.

In an embodiment of the present disclosure, there is provided a process for preparing the trapping device for trapping microbes as disclosed herein, wherein molding the polymeric structure is carried out using a mold of a breadth:length ratio in a range of 1:50 to 10:200 to obtain the device.

In an embodiment of the present disclosure, there is provided a process for preparing the trapping device for trapping microbes as disclosed herein, wherein the suture is added to the first solution prior to vacuum drying.

In an embodiment of the present disclosure, there is provided a process for preparing the trapping device for trapping microbes as disclosed herein, wherein the porogen is selected from sodium chloride, paraffin spheres, sugar, or gelatin. In another embodiment of the present disclosure, the porogen is sodium chloride.

In an embodiment of the present disclosure, there is provided a process for preparing the trapping device for trapping microbes as disclosed herein, wherein the suture is obtained by extrusion or melt spinning of a biocompatible material selected from polyurethane, polyethylene, polypropylene, polycaprolactone, polylactic acid, polystyrene, silicone, polysaccharide, or combinations thereof.

In an embodiment of the present disclosure, there is provided a process for preparing the trapping device for trapping microbes as disclosed herein, the process comprising: a) mixing a one or more of a biocompatible polymer selected from polycaprolactone, polylactic acid, or combinations thereof with a porogen selected from sodium chloride, paraffin spheres, sugar, or gelatin optionally in the presence of silver nanoparticles, an antimicrobial agent, or an additive to obtain a first solution; b) adding a suture to the first solution followed by vacuum drying the first solution to obtain a polymeric structure; and c) molding the polymeric structure using a mold of a breadth:length ratio in a range of 1:50 to 10:200 to obtain the device.

In an embodiment of the present disclosure, there is provided a process for preparing the trapping device for trapping microbes as disclosed herein, the process comprising: a) mixing a one or more of a biocompatible polymer selected from polycaprolactone, polylactic acid, or combinations thereof with a porogen selected from sodium chloride, paraffin spheres, sugar, or gelatin optionally in the presence of silver nanoparticles, an antimicrobial agent, or an additive to obtain a first solution; b) adding a suture to the first solution followed by vacuum drying the first solution to obtain a polymeric structure; and c) molding the polymeric structure using a mold of a breadth:length ratio in a range of 1:50 to 10:200 to obtain the device; and wherein the suture is left along as an extension to obtain the string of the device.

In an embodiment of the present disclosure, there is provided use of the trapping device for trapping a microbe, wherein the microbe is selected from bacteria, fungi, or virus; and the microbe is selected from Escherichia coli, Pseudomonas aeruginosa, Helicobacter pylori, Salmonella species, Vibrio cholera, Klebsiella, Staphylococcus, or Enterococcus.

In an embodiment of the present disclosure, there is provided use of the trapping device for treating water by trapping and killing microbes in water.

In an embodiment of the present disclosure, there is provided use of the trapping device for treating or preventing a disease or a condition or an infection caused by a microbe.

In an embodiment of the present disclosure, there is provided use of the trapping device, for treating a urinary tract infection by preventing or inhibiting growth of microbes or by killing microbes.

In an embodiment of the present disclosure, there is provided use of the trapping device in a packaged material to prevent or inhibit growth of microbe selected from bacteria, virus, or fungi.

In an embodiment of the present disclosure, there is provided a method of treating or preventing a disease or a condition or an infection caused by a microbe, the method comprising: a) inserting the device comprising one or more of a porous polymeric structure with a proximal end and a distal end, wherein the proximal end and the distal end is optionally provided with a hole or core on the longitudinal direction with a delivery tool into urinary bladder through urethra; b) releasing the device into the urinary bladder and removing the delivery tool; c) allowing the device to trap the microbes; and d) reinserting the delivery tool into the urinary bladder and retracting the device.

In an embodiment of the present disclosure, there is provided a method of treating or preventing a disease or a condition or an infection caused by a microbe, wherein the delivery tool is a catheter or the device is deposited via direct visualization into the lumen with an endoscope, and the retrieval tool is a hook type device, a magnetic retrieval device, or an endoscope with a grasper.

In an embodiment of the present disclosure, there is provided a method of treating or preventing a disease or a condition or an infection caused by a microbe, wherein the device is retrieved by directly pulling out the device or by pulling the string of the device.

In an embodiment of the present disclosure, there is provided a method of treating or preventing a disease or a condition or an infection caused by a microbe, the method comprising: a) inserting the device comprising one or more of a porous polymeric structure with a proximal end and a distal end, wherein the proximal end and the distal end is optionally provided with a hole or core on the longitudinal direction with a delivery tool into urinary bladder through urethra; b) releasing the device into the urinary bladder and removing the delivery tool; c) allowing the device to trap the microbes; and d) reinserting the delivery tool or a retrieval tool into the urinary bladder and retracting the device.

In an embodiment of the present disclosure, there is provided a method of treating or preventing a disease or a condition or an infection caused by a microbe as disclosed herein, wherein the delivery tool is a catheter or the device is deposited via direct visualization into the lumen with an endoscope, and the retrieval tool is a hook type device, a magnetic retrieval device, or an endoscope with a grasper.

In an embodiment of the present disclosure, there is provided a method of treating or preventing a disease or a condition or an infection caused by a microbe, the method comprising: a) inserting the device comprising one or more of a porous polymeric structure with a proximal end and a distal end, wherein the proximal end and the distal end is optionally provided with a hole or core on the longitudinal direction with a delivery tool into urinary bladder through urethra; b) releasing the device into the urinary bladder and removing the delivery tool; c) allowing the device to trap the microbes; and d) reinserting the delivery tool or a retrieval tool into the urinary bladder and retracting the device, wherein the delivery tool is a catheter or the device is deposited via direct visualization into the lumen with an endoscope, and the device is retracted by directly pulling out the end of the device or by pulling the string of the device.

In an embodiment of the present disclosure, there is provided a device as disclosed herein, wherein the device is used for preventing or treating infection in the urinary tract.

In an embodiment of the present disclosure, there is provided a trapping device for trapping microbes as disclosed herein, the device is used for water purification by reduction of bacterial load in potable water containers. In another embodiment of the present disclosure, the device is used for food and drug preservation. In one another embodiment of the present disclosure, the device is used for environmental applications such as treating reservoirs with problematic bacterial overgrowth.

In an embodiment of the present disclosure, there is provided a trapping device for trapping microbes as disclosed herein, wherein the device prevent recurrent upper tract pyelonephritis, catheter-associated infections, and infection-related urinary stone disease, and to prevent the colonization of indwelling ureteral stents, nephrostomy tubes, and tunneled haemodialysis lines.

In an embodiment of the present disclosure, there is provided a trapping device for trapping microbes as disclosed herein, wherein the device is incorporated into standard indwelling clinical devices, including double-J ureteral stents, chronic bladder catheters, and indwelling venous and arterial catheters.

Although the subject matter has been described in considerable detail with reference to certain examples and implementations thereof, other implementations are possible.

### EXAMPLES

The disclosure will now be illustrated with working examples, which are intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

The forthcoming examples explain how the present disclosure provides a device comprising one or more polymeric structure and a process for preparing the device. The device comprises silver nanoparticles incorporated within the polymeric structure which exhibits the bactericidal activity. The device of the present disclosure is molded to definite and requisite shapes and sizes. The polymeric structure is made of a biocompatible polymer selected from polycaprolactone, polylactic acid, or combinations thereof.

### Materials and Methods

For the purpose of the present disclosure, the following raw materials with the specified grades/brands were used.
a. Polycaprolactone—Sigma-Aldrich (PCL 80,000 g/mol);
b. Silver nanoparticles (AgNPs)—Sigma-Aldrich (with size <100 nm);
c. Copper nanoparticles (CuNPs)—purity 98% with size <100 nm;
d. Porogen—sodium chloride—(NaCl, Tata edible salt) sieved to the size range of 125-250 μm, 150-350 μm;
e. Luria Broth (LB)—Sigma-Aldrich; and
f. Escherichia. coli (DH5α) strain.

### Example 1

### Preparation of the Device

The process of preparation of the device comprising a porous polymeric structure with a proximal end and a distal end, wherein the proximal end and the distal end is optionally provided with a hole or core on the longitudinal direction is as follows.

In an example, 135 μl of 10% (w/v) polycaprolactone solution, was added to the salt bed containing sodium chloride to obtain a first solution. The first solution was vacuum dried and the well plate was kept for salt leaching in deionized water for three days to obtain a polymeric structure. After three days, the porous polymeric structure samples were taken out from the plate to obtain the device. The well plate is a mold of definite shape and size. The mold was of varying sizes with a breadth to length ratio in the range of 1:50 to 10:200 and was selected to obtain a device of requisite shape and size. The mold was selected in various shapes such as circular, square or any shape and hence the device also attained the molded shape.

Silver nanoparticles of varying concentration such as 0.05 mg/ml, 0.5 mg/ml, 5.0 mg/ml, or 15.0 mg/ml were prepared by mixing silver nanoparticles (AgNPs) in 10 ml of tetrafluoroethylene solution followed by probe sonication at 50 kHz for 20 min. 1 g of polycaprolactone was added with sodium chloride to obtain a first solution. A solution of silver nanoparticles was added to the first solution. The first solution was poured in 48 well plates and the polymeric structure was obtained by salt leaching method.

In another example, 135 μl of 10% (w/v) polycaprolactone solution, containing suspended AgNPs, was added to the salt bed containing sodium chloride to obtain a first solution. The first solution was vacuum dried and the well plate was kept for salt leaching in deionized water for three days to obtain a polymeric structure. After three days, the porous polymeric structure samples were taken out from the plate to obtain the device. The well plate is a mold of definite shape and size. The mold was of varying sizes with a breadth to length ratio in the range of 1:50 to 10:200 and was selected to obtain a device of requisite shape and size. The mold was selected in various shapes such as circular, square or any shape and hence the device also attained the molded shape.

The porous polymeric structures were also generated by lyophilizing chitosan.

In another example, a suture was obtained by extrusion or melt spinning of a biocompatible polymer selected from polycaprolactone, polylactic acid, or combinations thereof. The suture was suspended in the first solution and was vacuum dried followed by salt leaching in deionized water for three days to obtain a polymeric structure. After three days, the porous polymeric structure with the suture was taken out from the plate/mold to obtain the device.

In yet another example, a string which is an extended suture was placed in the first solution and was vacuum dried followed by salt leaching in deionized water for three days to obtain a polymeric structure. After three days, the porous polymeric structure with the suture and the string was taken out from the mold/plate to obtain the device. Medical grade inorganic polymer such as silicones was also used to obtain a string. Also, a polysaccharide such as silicone was also used to prepare a string.

The device comprising the polymeric structure was obtained with varying pore sizes in a range of 125-250 μm and 150-350 μm based on the size of the porogen used.

Scanning electron microscopic images of the device comprising the polymeric structures were obtained using Zeiss Ultra 55. The porous polymer samples of the present disclosure were coated with 10 nm gold coating for 2 minutes using a JEOL Smart Coater. The electron micrographs were captured at 3 keV and the corresponding elemental analysis were captured at 10 keV using a SEM-EDS system Zeiss Ultra 55 (Scanning Electron microscopic—Energy dispersive X-ray analysis). FIG. 1A depicts the neat polycaprolactone solution and porous polycaprolactone polymeric structures fabricated through the salt leaching method. The fabricated polymeric structures with different pore sizes, as shown in FIGS. 1B and 1C of varying pore sizes i.e., (B1 and B2) pore size 125-250 μm, and (C1 and C2) pore size 125-350 μm were obtained. The pore formation was confirmed through SEM micrographs on both sides of the structures. It could be observed that the pore size corresponded to the porogen size, and the pores were interconnected.

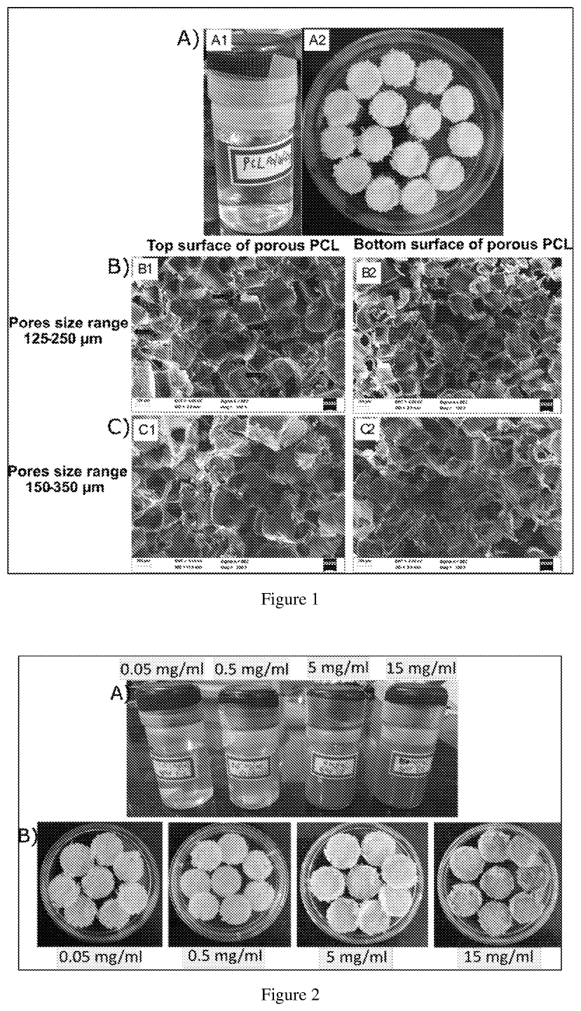

FIG. 2A depicts the polycaprolactone solution containing well-dispersed AgNPs. An increase in silver concentration from left to right was reflected by the change in the color of the solution, and the same was observed in optical micrographs of the obtained porous polycaprolactone polymeric structures as shown in FIG. 2B. The increase in silver concentration led to a color change from white to brownish.

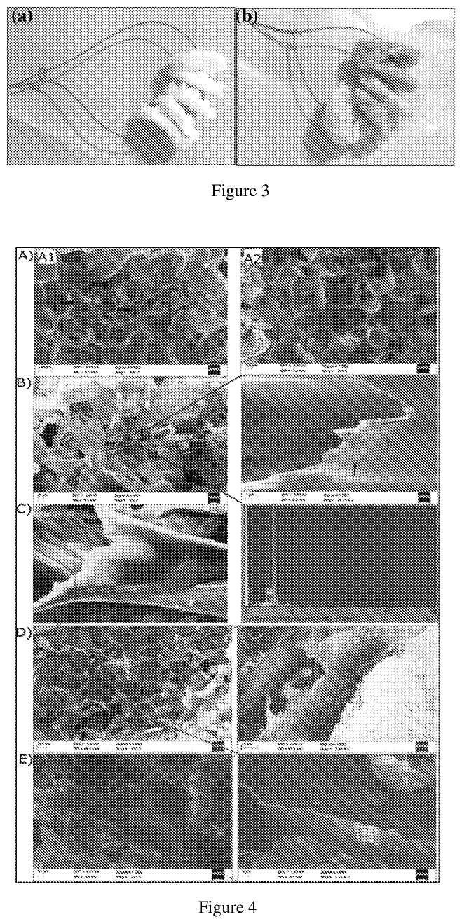

FIG. 3A depicts the device of the present disclosure comprising series of the polymeric structures comprising polycaprolactone. FIG. 3B illustrates series of the polymeric structures comprising polycaprolactone incorporated with silver nanoparticles (AgNPs).

### Example 2

### Mechanical Trapping of the Bacteria by the Device

### Bacterial Testing:

In vitro testing was performed to determine the bacterial trapping and subsequent killing ability of the device. E. coli inoculation cultures were prepared and standardized by adjusting the OD at 600 nm to 0.4. LB media was maintained at pH of 6.4. 200 μl E. coli inoculation was suspended into 1800 μL LB media and gently shaken. The as-prepared devices comprising the polymeric structure were sterilized under UV light for 15 min and then placed in the bacterial suspension with gentle rocking at 6 rpm at 37° C. The OD value of the suspension was recorded at serial time points.

SEM micrographs on both sides of the device comprising polycaprolactone with AgNPs are shown in FIG. 4A (4A1) the top surface and (4A2) the bottom surface. Cross-sectional images revealed that AgNPs were embedded on the walls of interconnected pores. In FIG. 4B, the arrow points denotes that the AgNPs are embedded on the walls of the polymeric structure. This observation was validated through energy dispersive spectroscopy by identifying the silver peak corresponding to 3 keV (FIG. 4C).

Trapped E. coli were seen in interconnected pores, as shown in FIG. 4D. The presence of AgNPs in the device killed the bacteria trapped in the device, which was corroborated through SEM analysis. FIG. 4D shows the entrapped E. coli within the interconnected pores, which were intact, whereas morphologically disrupted E. coli was seen in FIG. 4E at a silver concentration of 0.5 mg/ml. FIG. 4E clearly indicated that the bacteria were killed in the device and thereby confirming the device was capable of trapping and killing the bacteria.

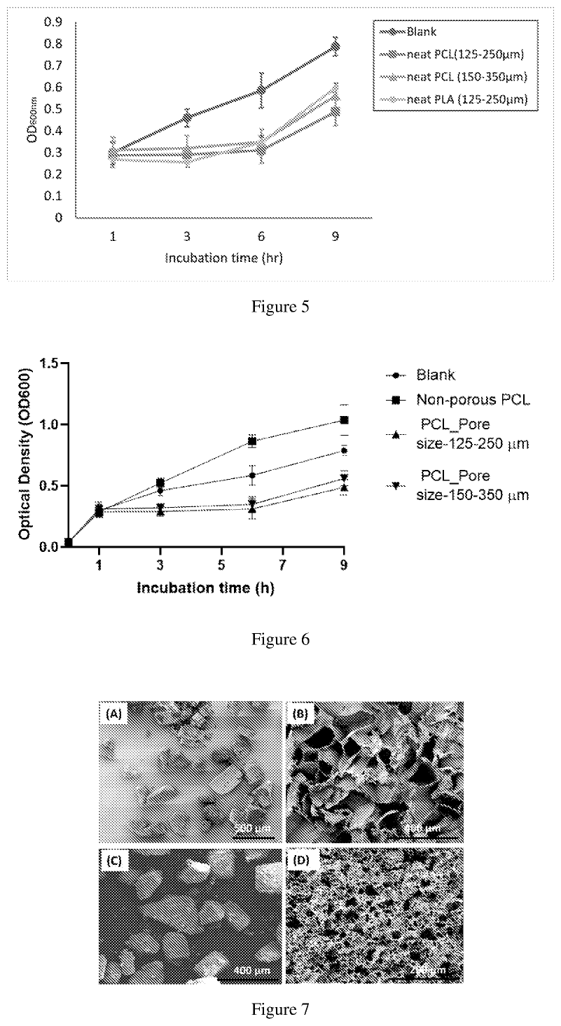

Accordingly, the optical density of the bacterial suspensions were measured to understand the concentration of bacteria trapped and killed by the device. Bacterial concentration in suspension was assessed at different durations of incubation. The OD value at 600 nm was used as a measure of the suspended bacterial load (FIG. 5). Initially (at 1 h), no significant difference was observed between bacterial concentration in the medium alone and the suspension containing the device comprising the porous polycaprolactone. However, over the next 3-9 hours, the device comprising the porous polycaprolactone (neat PCL) was effective in reducing the suspended bacterial load. There was no significant difference in the OD values for the two different ranges of pore size of the foams tested. Moreover, the same was observed for the device comprising the porous polylactic structures (neat PLA-FIG. 5). The OD measurements confirmed that the motile E. coli was observed to be trapped within interconnected pores of the device (FIG. 4D).

Further experiments were carried out to understand the effect of pore size on trapping of bacteria. FIG. 6 shows the device having varied pore sized porous polymeric structure (polycaprolactone, porous PCL) and their capability to reduce the E. coli load in a solution. It could be observed that the bacterial trapping capability of 125-250 μm porous PCL was greater than that of 150-350 μm pore sized porous PCL. Therefore, it could be understood that porous PCL with smaller pores trapped as many bacteria as possible. Thus, porous PCL was prepared by creating smaller pores, as illustrated in FIG. 7.

NaCl crystals (porogen) (125-250 μm) were crushed to a smaller sized salt crystals having pore size of 45-63 μm, and was used as porogen to obtain porous PCL with smaller pores. FIG. 7A shows uncrushed 125-250 μm range NaCl crystals with corresponding porous PCL (FIG. 7B), whereas FIGS. 7C and 7D show 45-63 μm range NaCl crystals with corresponding porous PCL. As shown in FIG. 7D, the smaller crystal size resulted in smaller pores with a rougher surface. This rough texture aided in physically avoiding bacterial attachment.

The bacterial trapping ability of an as-molded polycaprolactone (PCL) with no pore structure (0% porosity) and a porous polycaprolactone structure (with porosity in a range of 70-98% was tested for 9 hours. When compared to PCL structure with 0% porosity, the porous structure (pore size ranges from 125-250 μm) significantly reduced the bacterial load from the suspension. Therefore, it was confirmed that the porous structure of the device facilitated trapping the microorganisms.

The device of the present disclosure was fabricated using molds of varying shapes and size and any metallic or polymer molds. Additionally, the device was incorporated with nanoscale surface topography (using a reactive ion etching system and another way of generative nanostructures on and within the scaffold surface) or silver nanoparticles, to minimize the growth of suspended bacterial cells. Antibacterial chemicals/agents such as metal particles (Au, Cu, etc.) metal oxide particles (CuO, ZnO, TiO₂, Fe₃O₄, etc.), antibiotics, cationic polymers, and other antibacterial agents were embedded within the polymeric structure or grafted onto the surface of the walls of the device. Toxins, chelating agents such as polymyxin B and attractants were also embedded in the device for local activity/sustained release to minimize the proliferation of trapped motile bacteria as well as to attract and neutralize non-motile bacteria as well.

### Nanotextured Porous PCL Through Reactive Ion Etching.

PCL porous samples were etched using oxygen (O₂) plasma inside a custom-built RIE plasma etching chamber equipped with a 13.56 MHz RF generator (Berthold, Germany) in a capacitively coupled (CCP) configuration. The 300 W RF generator capable of delivering up to 300 W max power at 13.56 MHz was used. Nanotexturing was achieved using oxygen plasma struck by introducing 99.9% pure oxygen into the reactor chamber via a showerhead which ensured even distribution of the gas. The gas flow rate was controlled using a mass flow controller (Alicat Corporation, USA), and the pressure inside the chamber was maintained with the help of a turbomolecular pump (Edwards, UK) backed by a rotary vane pump (HPVT technologies, India). The distance between the electrodes was maintained at 10 cm. Samples were placed on clean silicon wafers and etched under different values of radio frequency (RF) power, gas flow rates, and chamber pressures. A Capacitively Coupled Plasma (CCP) reactor was used in which the feed gas was fed directly between two parallel plates (electrodes) while the chamber was kept at a constant pressure. The feed gas was ionized when an RF voltage is put between the electrodes, and the resulting plasma was employed for various etching procedures. The bottom electrode had a negative voltage focused on the so-called sheath layer, where ions were accelerated according to the negative potential of the lower electrode (commonly referred to as the direct current (DC) bias) as well as the ion charge. As a result, the ions accumulate kinetic energy, which then dissipate when they strike the lower electrode (or sample to be etched). Ion energy was frequently required to activate an etching process, which is referred to as ion-driven etching. Pressure and gas flow have direct effects on DC bias in this system, however, RF power had the greatest impact on DC bias, and increasing RF power increased DC bias and, thus, ion energy.

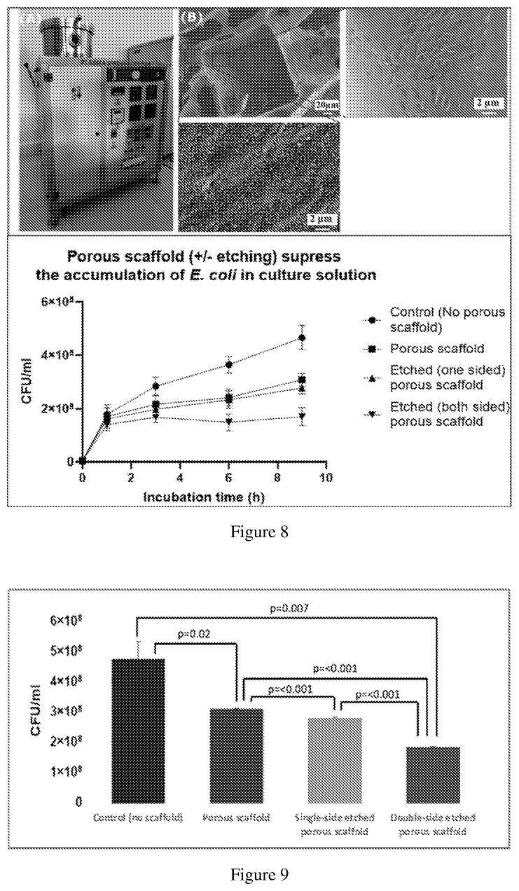

The formation of random nanoscale features was ascribed to preferential etching by oxygen plasma directed by the co-deposition of substrate nanoparticles randomly sputtered from the cathode. Such co-deposition resulted in the formation of micro-masks on the PCL surface, and the area directly beneath such masks had a slower etch rate than the pristine regions of PCL. This process of micro-masking was reminiscent of the generation of nanotextures on surfaces. The photograph of RIE system is shown in FIG. 8(A).

Parameters and results: RF Power: 30 W, Gas flow rate: 40 sccm, Chamber Pressure: 0.05 mbar, Time: 5 min. The curvy and uneven surface of the macroporous PCL surface influenced ion interaction, resulting in different morphology and density of nanotextures at different locations, as shown in FIG. 8(B). Even though the nanotextures were not uniformly created over the porous PCL, a slight reduction in bacterial load was observed compared to a neat porous PCL sample till 9 h. However, this was further improved by optimising the aforementioned parameters and reducing the bacterial load from the suspension (FIG. 8C). A dose-dependent effect of RIE etching confirmed that porous PCL etched on both sides cleared viable E. coli from solution more effectively than porous PCL etched on one side only or porous PCL alone. Mean colony forming unit (CFU) at 9 h incubation (3 replicates) were compared between individual groups with a one-tailed t-test demonstrating a significant dose-dependent effect as shown in FIG. 9. The both sided etched PCL showed significant bacterial load from suspended media.

### Incorporation of Silver Nanoparticles

Despite the initial suppression, the rapid growth of the bacterial load in solution was seen at longer time periods (24 h). This further implied that sequestration alone had limited efficacy in reducing the overall bacterial load in solution and that additional measures were required to block the ongoing proliferation of the trapped bacteria. Hence the device was incorporated with other materials which includes but not limited to antimicrobial agent selected from metal particles, metal oxide particles, antibiotics, cationic polymers, or combination thereof, or an additive selected from chelating agent, toxins, attractants, or combinations thereof. In particular, the device comprised a chelating agent such as polymyxin B which potentially bound endotoxin for inhibiting or killing the bacterial growth.

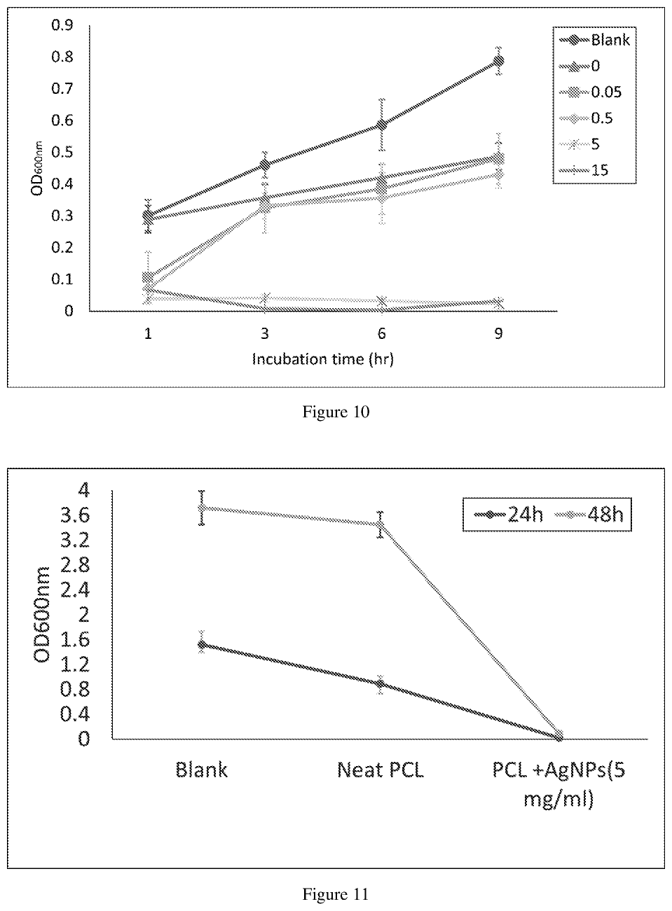

Thus, in order to further improve the bactericidal activity of the device, the device comprising a porous polymeric structure with varying concentration of silver nanoparticles were fabricated as explained in Example 1 above and were tested. A dose-dependent effect of incorporated silver nanoparticles (AgNPs) over the presence of porous polycaprolactone was seen on bacterial load in solution (FIG. 10), which was due to the combination of local effects on trapped bacteria and release of AgNPs into solution over time. It could be observed that the higher concentration of AgNPs killed almost all of the bacteria and thereby confirming the device effectively trapped and killed bacteria. The porous structures of the device facilitated trapping the bacteria and the presence of AgNPs provided the bactericidal effect to the device.

Further the bactericidal effect was studied for extended period up to 48 hours to provide a comparative study on the device comprising the polycaprolactone only and with AgNPs. FIG. 11 depicts the optical density values of the E. coli suspension in contact with the device comprising porous polycaprolactone alone and the device with porous polycaprolactone loaded with silver nanoparticles for a time period of 24 hours and 48 hours. It could be noted that the device comprising polycaprolactone with silver nanoparticles exhibited higher mechanobactericidal effect compared to the device with polycaprolactone only.

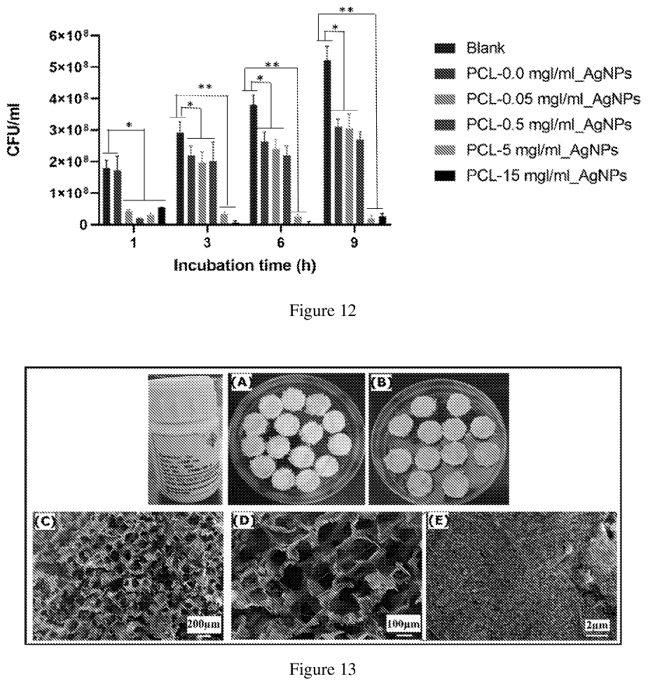

Effect of incubation time on varying AgNPs dose was also studied and it was found that the higher concentration (5 mg/ml and 15 mg/ml) of AgNPs were effective in nearly complete reduction in bacteria in suspension (FIG. 12) under prolonged incubation time.

Loading of Antimicrobial Nanoparticle into Porous PCL

Instead of AgNPs, copper nanoparticles (CuNPs) with 5 mg/ml of concentration were loaded within the macroporous PCL structure. The color change of PCL foam from white to light bluish was observed as seen in FIG. 13(A,B). The electron micrographs of CuNPs loaded porous PCL are shown in FIG. 13(C-E). The CuNPs embedded on the wall of PCL are depicted in FIG. 13E. Thus, it could be confirmed that the salt leaching method of the present disclosure was capable and flexible enough to load a wide range of antibacterial agents.

Release of Silver and Copper Nanoparticles in Solution from Doped Porous PCL

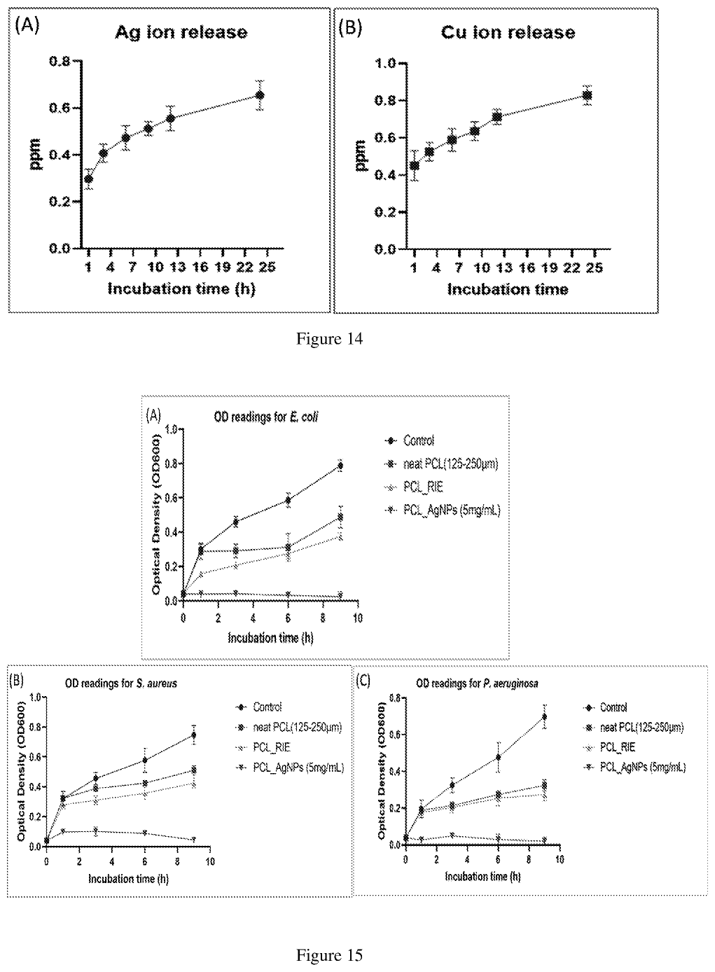

The release of silver and copper nanoparticles in solution from doped porous PCL was detected and is explained herein. For a 5 mg/mL concentration of Ag and Cu nanoparticles loaded within the macroporous PCL, the amount of silver and copper ions was measured. One sample of each was dipped in PBS and was shaken at 6 rpm for various periods at 37° C. The samples were then removed, and the solution was filtered through filter paper. The principle of absorption ability of these ions against a specific wavelength was used to detect the percentage of silver/copper ions using atomic absorption spectroscopy and the results are shown in FIGS. 14A and B.

Further the device of the present disclosure i.e., the porous PCL incorporated with silver nanoparticles was found to be efficient in reducing both motile and non-motile bacterial concentration in solutions as shown in FIG. 15 (A, B and C) which illustrate the optical density values for (A) E. coli, (B) S. aureus, and (C) P. aeruginosa bacterial culture suspensions in contact with porous PCL (125-250 μm pore sizes) either neat or etched on one side or loaded with AgNP concentrations of 5 mg/mL.

### Example 3

### Method of Treating Urinary Tract Infection

The device of the present disclosure could treat or prevent the urinary tract infection. The device was inserted in to the urinary bladder through urethra using a delivery tool and the device was released inside the urinary bladder. The delivery tool was removed and then the device was allowed to trap the microbes in the urinary bladder. The device was then retracted from the urinary bladder using the delivery tool and found with bacterial load, thereby reducing the bacterial load in the bladder and treating the urinary tract infection. The delivery tool used herein is the catheter or the device was deposited via direct visualization into the lumen with an endoscope. The delivery tool used to retrieve the device also includes but not limited to a hook type device, a magnetic retrieval device, or an endoscope with a grasper.

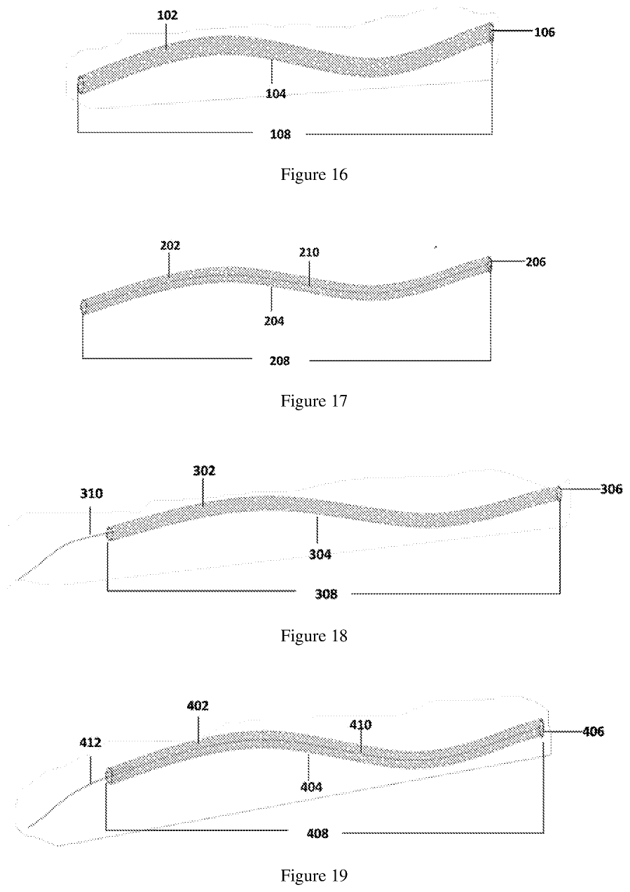

FIG. 16 depicts the prototype device of the present disclosure comprising the porous polymeric structure (102) incorporated with silver nanoparticles. FIG. 16 illustrates the device having walls (104) of the porous polymeric structure which has silver nanoparticles incorporated in it. The device has a hole or core (106) in the longitudinal direction from the proximal end to the distal end (108).

FIG. 17 also depicts the device comprising the porous polymeric structure (202) incorporated with silver nanoparticles with the suture (210) embedded in the polymeric structure. FIG. 17 illustrates the device having walls (204) of the porous polymeric structure and the hole or core (206) in the longitudinal direction from the proximal end to the distal end (208).

FIG. 18 illustrates the device comprising the molded porous polymeric structure (302) with the string (310) and the device has silver nanoparticles embedded in it or the trapped on the walls (304) of the polymeric structure. The device has proximal and distal ends (308) in the longitudinal direction and is optionally provided with hole or core (306).

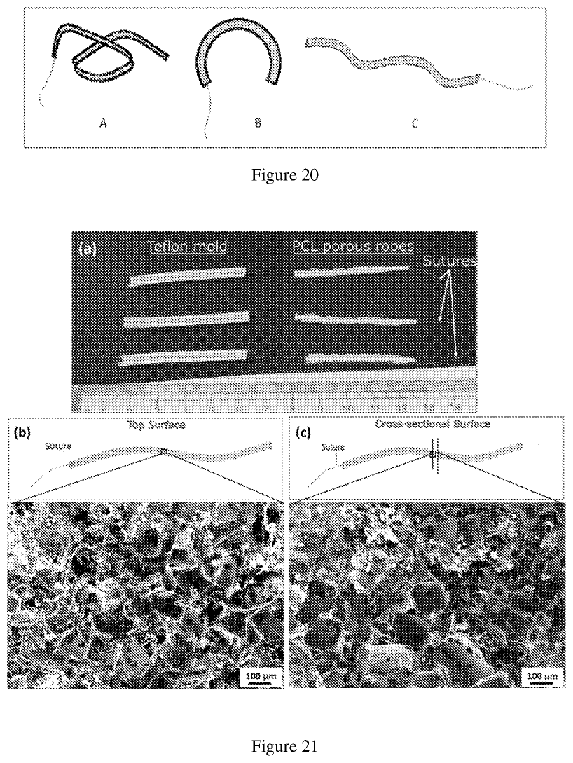

FIG. 19 depicts one form of the device of the present disclosure comprising the porous polymeric structure (402) with a string (412) and a suture (410). The walls (404) of the device is also porous and the device is optionally provided with hole or core (406) at the proximal and distal ends (408) in the longitudinal direction. The suture provided support to the device and the string is an extension of the suture useful in retracting the device. Some of the representative images of the further complex or folded structure of the device are shown in FIG. 20 (A, B and C).

### Fabrication of Porous PCL Ropes

For making PCL porous rope, a Teflon tube with an inner diameter of 2 mm was used as a mold. The Teflon tube was cut in half so that the salt and PCL solution could be poured manually. This method allowed to create a long porous rope, and the PCL solution also penetrated to the bottom layer of the salt bed. The same procedure was used to create PCL porous rope. Before pouring the PCL solution, the biomedical sutures were placed in the salt bed. FIG. 21(A) depicts optical photographs of a Teflon mold (Diameter: 2 mm, Length: 4 cm) and PCL porous ropes with sutures extracted from the mold. Furthermore, top, and cross-sectional SEM images of one of the PCL ropes were captured (FIGS. 21 B, C). There appeared to be no significant change in the morphology of the top and cross-sectional surfaces, and the porous nature of the rope was confirmed. This demonstrated that the salt leaching process was capable of successfully producing long porous ropes.

In an instance, the fabricated trapping device of the present disclosure could be delivered by a various mechanism such as the device is placed endoscopically into the bladder lumen or directly inserted via the urethra inside a mesh sack attached to a string for easy retrieval and exchange. The combined mechanical approach of sequestration and local bactericidal effects reduced the urinary tract infections (UTIs) rates by reducing the chronic bacterial load (colonization) while avoiding the side effects and risks of resistance with recurrent prophylactic and therapeutic antibiotic use.

Beyond standard recurrent UTIs, the device of the present disclosure provided a mechanical approach which was helpful to prevent recurrent upper tract pyelonephritis, catheter-associated infections, and infection-related urinary stone disease, as well as prevented the colonization of indwelling ureteral stents, nephrostomy tubes, and tunneled hemodialysis lines.

The device comprising the porous polymeric structures are flexible and can easily be incorporated into standard indwelling clinical devices, including double-J ureteral stents, chronic bladder catheters, and indwelling venous and arterial catheters. Thus, the device of the present disclosure is easy to use and involves cost-effective fabrication techniques which makes this device a highly marketable and competitive antibiotic alternative.

### ADVANTAGES OF THE PRESENT DISCLOSURE

The present disclosure discloses a trapping device comprising a porous polymeric structure with porosity in a range of 70-98% and is optionally provided with a hole or a core in the longitudinal direction. The porous polymeric structure of the device mechanically traps the bacteria. The device of the present disclosure comprises silver nanoparticles which provide bactericidal effect. The present disclosure provides a mechanobactricidal traps for the microbes. The device of the present disclosure is adaptable to be fabricated to requisite shapes and sizes as based on its area of application. The device also comprises micro or nano scaled surface textures which can be optimized to allow for maximum exposure to microbes and to maximize microbial cell death. The device further comprises an antimicrobial agent or an additive selected from antimicrobial agent, toxins, attractants, thereby the device can be customized to sequester and neutralize pathogenic motile and non-motile bacteria and decrease the overall suspended bacterial load. The present disclosure also provides a cost-effective process for fabricating the device and the process shall be carried out at room temperatures, thereby making the process easily scalable. The room temperature leaching process of the present disclosure allows for the incorporation of materials such as chelating agents, toxins, attractant, or antimicrobial agents within the scaffolds of the device. The device of the present disclosure shall be molded to any shape and thereby providing a wide range of clinical and environmental applications. The present disclosure further provides an implantable or an insertable device. The device of the present disclosure is useful in preventing and inhibiting urinary tract infections, The device is especially useful for treating bacterial infections of patients having antibiotic resistance. The device of the present disclosure is also useful in the prevention of other clinical infections such as venous and arterial vascular catheter-related infections, food and drug preservation, where the biocompatible device shall be placed within shelf-stable solutions, in water purification for reduction of bacterial load in potable water containers, in environmental applications such as treating reservoirs with problematic bacterial overgrowth, and to areas in which bacteria needs to be removed.

I/We claim:

1. A trapping device for trapping microbes, the device comprises a porous polymeric structure with a proximal end and a distal end, wherein the proximal end and the distal end is optionally provided with a hole or core on the longitudinal direction.

2. The device as claimed in claim 1, wherein the polymeric structure has a breadth to length ratio in the range of 1:50 to 10:200, a porosity in the range of 70 to 98% and pore size in the range of 10 to 500 μm.

3. The device as claimed in claim 1, wherein the device comprises one or more of the polymeric structures and is arranged linearly or in complex folded shape for deployment.

4. The device as claimed in claim 1, wherein the polymeric structure is incorporated with silver nanoparticles.

5. The device as claimed in claim 1, wherein the device comprises one or more antimicrobial agent or an additive, embedded within the polymeric structure or grafted on to the surface of the walls of the trapping device.

6. The device as claimed in claim 5, wherein the antimicrobial agent is selected from metal particles, metal oxide particles, antibiotics, cationic polymers, or combinations thereof; and the additive is selected from chelating agent, toxins, attractants, or combinations thereof.

7. The device as claimed in 6, wherein the chelating agent is polymyxin B capable of binding to endotoxin.

8. The device as claimed in claim 6, wherein the metal particles is selected form Au, Cu, or combinations thereof; and the metal oxide particles selected from CuO, ZnO, TiO₂, Fe₃O₄, or combinations thereof.

9. The device as claimed in claim 1, wherein the device has a surface texture in microscale, nanoscale, or combination of micro-nano scale; and the surface texture is achieved by means of top-down or bottom-up approach.

10. The device as claimed in 9, wherein the surface texture ranges from 10 nm to 1000 nm in height and in spacing.

11. The device as claimed in 9, wherein the surface texture has been optimized to allow for the application of insonation to maximize bacterial cell death at the surface-urine interface.

12. The device as claimed in claim 1, wherein the polymeric structure is made of a biocompatible polymer selected from polycaprolactone, polylactic acid, chitosan, or combinations thereof.

13. The device as claimed in claim 1, wherein the device comprises a suture inserted between the proximal end to the distal end.

14. The device as claimed in claim 13, wherein the suture is a biocompatible material selected from polyurethane, polyethylene, polypropylene, polycaprolactone, polylactic acid, polystyrene, silicone, polysaccharide, or combinations thereof.

15. The device as claimed in claim 13, wherein the suture has breadth and length in a ratio range of 0.2:50 to 0.5:200; and the suture has the length in a range of 5 to 20 cm, and the breadth in a range of 0.2 to 0.5 mm.

16. The device as claimed in claim 1, wherein the device comprises a string.

17. The device as claimed in claim 16, wherein the string is an extension of the suture.

18. The device as claimed in claim 1 is an implantable or an insertable device.

19. The device as claimed in claim 1, wherein the device comprises a delivery tool at the proximal end adapted to deliver the device.

20. The device as claimed in claim 1, wherein the device is a mechanical trap.

21. The device as claimed in claim 1, wherein the microbe is selected from bacteria, fungi, or virus.

22. The device as claimed in claim 21, wherein the microbe is selected from Escherichia coli, Pseudomonas aeruginosa, Helicobacter pylori, Salmonella species, Vibrio cholera, Klebsiella, Staphylococcus, or Enterococcus.

23. A process for preparing the device as claimed in claim 1, the process comprising:
a) mixing one or more of a biocompatible polymer selected from polycaprolactone, polylactic acid, or combinations thereof, with a porogen optionally in the presence of silver nanoparticles, an antimicrobial agent, or an additive to obtain a first solution;
b) vacuum drying the first solution to obtain a polymeric structure; and
c) molding the polymeric structure to obtain the device.

24. The process as claimed in claim 23, wherein the biocompatible polymer is chitosan, which upon freeze drying, result in the polymeric structure.

25. The process as claimed in claim 23, wherein the suture is added to the first solution prior to vacuum drying.

26. The process as claimed in claim 23, wherein the porogen is selected from sodium chloride, paraffin spheres, sugar, or gelatin.

27. The process as claimed in claim 23, wherein the suture is obtained by extrusion or melt spinning of a biocompatible material selected from polyurethane, polyethylene, polypropylene, polycaprolactone, polylactic acid, polystyrene, silicone, polysaccharide, or combinations thereof.

28. The process as claimed in claim 25, wherein the suture is left along as an extension to obtain the string of the device.

29. Use of the device as claimed in claim 1 for trapping a microbe.

30. Use of the device as claimed in claim 1 in treating water.

31. Use of the device as claimed in claim 1 for treating or preventing a disease or a condition or an infection caused by a microbe.

32. Use of the device as claimed in claim 1 for treating a urinary tract infection by preventing or inhibiting growth of microbes or by killing microbes.

33. Use of the device as claimed in claim 1 in a packaged material to prevent or inhibit growth of microbes.

34. The use as claimed in anyone of the claims 28 to 32, wherein the microbe is selected from bacteria, virus, or fungi.

35. A method of treating or preventing a disease or a condition or an infection caused by a microbe, the method comprising:
a. inserting the device as claimed in claim 1 with a delivery tool into urinary bladder through urethra;
b. releasing the device into the urinary bladder and removing the delivery tool;
c. allowing the device to trap the microbes; and
d. reinserting the delivery tool or a retrieval tool into the urinary bladder and retracting the device.

36. The method as claimed in claim 34, wherein the delivery tool is a catheter or the device is deposited via direct visualization into the lumen with an endoscopic cytoscope, and the retrieval tool is a hook type device, a magnetic retrieval device, or an endoscope with a grasper.

37. The method as claimed in claim 34, wherein the device is retracted by directly pulling out the end of the device or by pulling the string of the device.
